SEQUENCE LISTING (52 sequences)

SEQ ID NO: 1 - protein, 237 aa, Mus montanus
PMYLFLGNLSFLDLSFTTSSIPQLLHNLSGRDKTISYVGCVVQLFLFLGLGGVECLLLAVMAYDRFVAVCKPLHYTVIMSSRLCLGLVSVAWGCGMANSLVMSPVTLQLPRCGHNKVDHFLCEMPALIRMACVNTVAIEGTVFVLAVGIVLSPLVFILVSYGHIVRAVFRIQSSSGRHRIFNTCGSHLTVVSLFYGNIIYMYMQPGSRSSQDQGKFLTLFYNIVTPLLNPFIYSLRN

SEQ ID NO: 2 - protein, 237 aa, Mus montanus
PMYLFLGNLSFVEVCLTSTTVPKILVNTQTLSKDISYRGCLTQVYFLMVFAGMDNFLLTVMAFDRFVAICYPLNYTVIMNPRLCVLLVLLSWLIMFWVSLLHILLLKRLTFSSGTAVPHFFCELSQLLKATSSDTLVNIILLYVVTALLGIFPATGILYSYSQIVSSLLRMSSSVGKSKAFSTCGSHLCVVSLFYGTGLGVHLSSAMNHPSQGNMIASVMLHCGHPMLNPIIYTLRN

SEQ ID NO: 3 - protein, 237 aa, Mus montanus
PMYLFLGNLSFLEVWYTTAAVPKALAILLGRSQSISFISCLLQMYLVFSLGCTEYFLLVAMAYDRYVAICFPLHYTTIMSLKLCLSLVVLSWVLTMLHALLHTLLVVRLSFCSDNVIPHFSCEISALLKLACSNTHVNELVIFITGGLVIVTPFLLILGSYVQIFSSILKVPSARGIHKAFSTCGSHLSVVSLFYGTIIGLYLCPSANNSTVKDTVVALMYTVVTPMLNPFIYTLRN

SEQ ID NO: 4 - protein, 234 aa, Mus montanus
PMYLFLGNLSFIDVCHSTVTVPKMLRDTWSEEKLISFDACVTQMFFLHLFACTEIFLLTVMAYDRYVAICKPLQYMTVMNWKVCVLLAVALWAGGTIHSISLTSLTIKLPYCGPDEIDNFFCDVPQVIKLACTDTHIIEILIVSNSGLISVVCFVVLVVSYAVILVSLRQQISEGRRKALSTCAAHLTVVTLFLGHCIFIYSRPSTSLPEDKVVSVFFTAVTPLLNPFIYSLRN

SEQ ID NO: 5 - protein, 237 aa, Mus montanus
PMYLFLGNLSLLEIGYTCSVIPKMLQSLVSEARGISREGCATQMFFFTLFAISECCLLAAMAFDRYMAICSPLHYATRMSRGVCAHLAVVSWTVGCMVGLGQTNYIFSLDFCGPCEIDHFFCDLPPILALACGDTSHNEAAVFVVAILCISSPFLLIVASYGRILAAVLVMPSPEGRRKALSTCSSHLLVVTLFYGSGSVTYLRPKASHSPGMDKLLALFYTVVTSMLNPIIYTLRN

SEQ ID NO: 6 - protein, 236 aa, Mus montanus
PMYLFLGNLSFLEILYTSTVVPKMLEGFLQVAAISVTGCLTQFFIFGSLATAECFLLAVMAYDRFLAICYPLRYPLLMGPRWCMGLVVTAWLSGFMVDELVVVLMAQLRFCGSNRIDHFYCDFMPLVVLACSDPRVAQVTTFVLSVVFLTVPFGLILTSYARIVVTVLRVPAGASRRKAFSTCSSHLAVVSTFYGTLMVLYIVPSAVHSQLLSKVFALLYTVVTPIFNPIIYSFRN

SEQ ID NO: 7 - protein, 237 aa, Mus montanus
PRYLFLGNLSLADIGISTTTIPQMVVNIQRKRKTISYAGCLTQVCFVLIFAGSENFLLAAMAYDRYAAICHPLRYTAIMNPHLCVLLVMISLSISTVDALLHSLMLLRLSFCTDLEIPHFFCELDQVITLACSDTLINNLLIYVTAGIFAGVPLSGIIFSYLHIVSSVLRMPSPGGVYKAFSTCGSHLSVVCLFYGTIFGVYISSAVTDSQRKGAVASVMYSVVPQMLNPIIYTLRN

SEQ ID NO: 8 - protein, 176 aa, Mus montanus
QALAYDRFVAICHPLHYLVIMSPRHCGFLTLVSFLLSLLDSQLHSFMTLNITSFKDVEISNFFCDPSQLLNLSCSNTFSDNIVKYFLGAFYGLFPISGILFSYYKIISSILRIPSLGGKYKAFSTCGSHLAVVCLFLVTASTVYLGSVASHSPRNDVVASLMYTVVTPMLNPFICS

SEQ ID NO: 9 - protein, 176 aa, Mus montanus
QALAYDRFVAICYPLHYMVIMNSRRCGLLILVSWIMSALHSLLQGLMMLRLSFCTDLEISHFFCELNHLVHLACSDTFLNEVVIYFAAVLLAGGPLAGILYSYCKIVSSIHAISSAQGKYKAFSTCASHLSVVSLFYCTSPGVYLSSAVTQNSHSTATASVMYSVVTPMLNPFICS

SEQ ID NO: 10 - protein, 176 aa, Mus montanus
QALAYDRFVAICHPLHYPRIMSQNLCFLLVVVSWVLSSANALLHTLLLARLSFLRGITLPHFFCDLSALLKLSSSDTTINQLAILTAGSAVVTLPFMCILVSYGHIGATILRRPSLKGICKALSTCGSHLSVVSVYYGAVIALYIVPSSNSTNDKDIAVSVLYTLVIPMLNPFICS

SEQ ID NO: 11 - protein, 176 aa, Mus montanus
QALAYDRFLAICYPLHYTVIMNPRLCGFSILVSFLLSLLDSQLHNLMILQITSFKDVEISSFFCDPSQLLNLSCSDNYSINTGKYVLFALYSFFPISGILFSYYKIISSILRIPSSGGKYKAFSTCGSHLAVFCLFLGTGTAVYFGSAVSHSPRENVVSSVMYTVVTPMLNPFICS

SEQ ID NO: 12 - protein, 176 aa, Mus montanus
QALAYDRFVAICHPLHYPTIMNPRFCGFLVLVSFLVSLLESQLHNLIALQFTTFKDVKIANFFCDPSQVLSLSCSGTFINIIVMYFVGALFGVFPISGILFSYYKIVSTILRIPSSGGKYKAFSTCGSHLSVVCLFYGTGFGVYLGSAVSHSSRKSAVASVMYTVVTPMLNPFICS

SEQ ID NO: 13 - protein, 168 aa, Mus montanus
GAIRFPLHNTTIMSPKLGLFLVVLSWVLTMFHAMLHTLLMARLCFCAENMIPHFFCDMSALLKLSCSNTHVNELVIFITAGLILLIPLVLILLSYGHIVSSILKVPSARGIHKTFSTCGSHLSVVSLFYGTVIGLYLCPSANNSTVKDTVMALMYTVVTPMLNPFICS

SEQ ID NO: 14 - protein, 176 aa, Mus montanus
QALAYDRFLAICHPLHYTAIMNPRLCGLLVLVCWILSVLHALLQSLMVLRLSFCRDIEIPHFFCELNQVVQLACFDNLLNDIVMNFALVLLATCPLAGILYSYSKIVSSIRAISSAQGKYKAFSTCASHLSVVSLFYCTGLGVYLSSAVSHSSRSSATASVMYTVVTPMLNPFICS

SEQ ID NO: 15 - protein, 119 aa, Mus montanus
HLCRLHLTVLKLACSDTLINNIVVFSMIIVLGVFPLSGILFSYSQIFSSILRISSDRGKYKVFSTCGSHLLVVSLFYGSSLGVYLSSVATLSSRMTLMASVMYTMVTPMLNPIIYTLRN

SEQ ID NO: 16 - protein, 159 aa, Mus montanus
PMYLFLGNLSFLEILYTSTVVPKMLEGFLQVAAISVTGCLTQFFIFGSLATAECFLLAVMAYDRFLAICYPLRYPLLMGPRWCMGLVVTAWLSGFMVDELVVVLMAQLRFCGSNRIDHFYCHFMPLVVLACSDPRVAQVTTFVLSVVPLTVPFGLILTS

SEQ ID NO: 17 - protein, 113 aa, Mus montanus
EDLCARLKRSRSDTTINEVGILTAGSAVVTLPFMCILVSYGHMGATILRRPSLKGMCKALSTCGSHLCVVSVYYGAVIALYIVPSSNSTNDKDIAVSVLYTLVIPMLNPFICS

SEQ ID NO: 18 - protein, 176 aa, Mus montanus
QALGYDRFVAMCHPGQYLVIMSPRHGGFLTLVSFLLSLLDSQLHSFMTLNITSFKDVEISNFFCDPSQLLNLSCSNTFSDNIVKYFLGAFYGLFPISGILFSYYKIISSILRIPSLGGKYKAFSTCGSHLAVVCLFLVTASTVYLGSVASHSPRNDVVASLMYTVVTPMLNPFICS

SEQ ID NO: 19 - protein, 176 aa, Mus montanus
QALAYDRFLAICHPLHYLVIMSPRHCGFLTLVSFLLSLLDSQLHSFMTLNITSFKDVEISNFFCDPSQLLNLSCSNTFSDNIVKYFLGAFYGLFPISGILFSYYKIISSILRIPSLGGKYKAFSTCGSHLAVVCLFLVTASTVYLGSVASHSPRNDVVASLMYTVVTPMLNPFICS

SEQ ID NO: 20 - protein, 176 aa, Mus montanus
QALAYDRFLAICHPRHYLVIMSPRHCGFLTLVSFLLSLLDSQLHSFMTLNITSFKDVEISNFFCDPSQLLNLSCSNTFSDNIVKYFLGAFYGLFPISGILFSYYKIISSILRIPSLGGKYKAFSTCGSHLAVVCLFLVTASTVYLGSVASHSPRNDVVASLMYTVVTPMLNPFICS

SEQ ID NO: 21 - protein, 112 aa, Mus montanus
PMYLFLGNLSFMDICFTTVVVPKMLANLLSETKGISYVGCLVQMYFFMAFGNTDSYLLASMAIDRLVAICNPLHYDVAMRPHRCLLMLLGSCTISHLHALFRVLLMSRLSFC

SEQ ID NO: 22 - protein, 119 aa, Mus montanus; identical to SEQ ID NO: 15

SEQ ID NO: 23 - protein, 141 aa, Mus montanus
WSLLESQLHSLRTLNMTSFRDVESSNLLCDPSQMLNLSCSNTFSDNIVKYFLGAFYGLFPISGILFSYYKIISSILRIPSLGGKYKAFSTCGSHLAVVCLFLVTASTVYLGSVASHSPRNDVVASLMYTVVTPMLNPFICS

SEQ ID NO: 24 - DNA, 711 bp, Mus montanus
cctatgtatttgttccttggcaacttgtccttcctggacctcagcttcaccaccagctccatcccccagctgctccacaacctgagtggccgtgacaagaccatcagctatgtgggctgcgtggtccagctcttcctgttcctgggcctgggtggagtggagtgtctactgctggccgtcatggcctatgacaggttcgtggccgtctgcaagcccctgcactacacggtgatcatgagttccaggctctgcctgggcttggtgtcagtggcctggggctgtggaatggccaactccttggtcatgtctccagtgaccctacaattaccccgctgcgggcacaataaggtggaccatttcctgtgtgagatgccagccctgatccgcatggcctgcgtcaacacagtggccatagaaggcactgtctttgtcctggccgtgggcatcgtgctgtctcccctggtcttcatcttggtgtcctatggccacatcgtcagggcggtgttcagaatccagtcgtcctcaggaagacacagaatcttcaacacctgtggctcccacctcaccgtggtctccctgttctacgggaacatcatctacatgtacatgcagccaggaagcaggtcctcccaggaccagggcaagttcctcaccctcttctacaacatcgtcacccccctcctgaaccccttcatctattccctcaggaat

SEQ ID NO: 25 - DNA, 711 bp, Mus montanus
cccatgtatttgttccttggtaacctgtcctttgtggaagtctgtttaacctccaccacggtccccaagatactggtgaacacgcagacactcagcaaagacatctcctacagaggctgccttactcaggtgtattttttaatggtttttgcaggtatggataatttccttctgactgtgatggcctttgaccgctttgtggccatctgctaccccctgaactatacggtcatcatgaaccccaggctctgtgtcctcctggtgctgctgtcttggctcatcatgttctgggtgtccttacttcacattctactcctgaagcgactgaccttctccagtggcactgcagtccctcatttcttctgtgaactgtctcagcttctcaaagcaaccagctctgacaccctcgtcaatatcatcttactgtatgtggtgactgccctgctgggtatcttccctgccactgggatcctctactcctactctcagatcgtctcttccttactgaggatgtcctcctctgtgggcaagtctaaagccttctccacctgtggttcccacctctgtgtggtctccttgttctatggaacaggtcttggggttcacctcagttctgccatgaaccatccttctcagggaaacatgattgcctccgtgatgttacactgtggtcaccccatgctgaaccccatcatctacaccctccggaac

SEQ ID NO: 26 - DNA, 711 bp, Mus montanus
cccatgtacttgtttcttggcaatctgtccttcctggaggtctggtacaccacggccgcagtgcccaaagccctggccatcctgctggggaggagccagagcatctccttcatcagctgcctcctgcagatgtacctggtcttctcgctgggctgcacggagtacttcctccttgtggccatggcttatgaccgctatgtggccatctgcttccccctgcactacaccaccatcatgagcctcaagctctgtctctccctggtggtgctgtcctgggtgctgaccatgctccatgccctgttgcacactctgcttgtggtcagattgtctttctgttcggacaatgtaatcccacacttttcctgtgaaatatctgctttattgaagctggcctgctccaacactcatgtcaatgaactggtgatatttatcacgggaggacttgttattgtcaccccatttctactcatccttgggtcctatgtacaaattttctcctccatcctcaaggtcccttctgctcgtggtatccacaaggccttctctacctgtggctcccacctctctgtggtgtcactgttctatgggacaattattggtctctatttatgtccatcagctaataattctactgtgaaagacactgtcgtggctctgatgtacacggtggtgactcccatgctgaaccccttcatctacaccctccgaaat

SEQ ID NO: 27 - DNA, 702 bp, Mus montanus
cccatgtacttgtttctcggtaacctgtcctttatcgatgtctgccactccactgtcactgtgcccaagatgctgagagacacctggtcagaggagaagctcatctcctttgatgcctgtgtgacccagatgttcttcctgcacctctttgcctgcacagagatcttcctcctcaccgtcatggcctatgatcgttatgtggccatctgtaaacccctgcagtacatgacagtgatgaattggaaggtatgtgtgctgctggctgtggccctctgggcaggaggaaccatccactccatatccctgacctccctcaccatcaagctgccctactgtggtcctgatgagattgacaacttcttctgtgacgtgccgcaggtgatcaaattggcctgcactgacacccacatcattgagatcctcatcgtctccaacagtgggctgatctccgtggtctgttttgtcgtccttgtggtgtcctatgcagtcatcctggtgagtctgcggcagcagatctccgagggcaggcggaaggccctgtccacctgtgcagcccacctcactgtggtcacactgttcctgggacactgcatcttcatctattcccgcccatccaccagcctcccagaggacaaagtggtgtctgtgtttttcactgctgtcacccctctgctaaaccccttcatctactccctccgaaat

SEQ ID NO: 28 - DNA, 711 bp, Mus montanus
cccatgtatttgttccttggtaacttgtctctcctagagattggctacacttgctctgtcatacccaagatgctgcagagtcttgtgagtgaggcccgaggaatctctcgggagggttgtgccacacagatgtttttctttacattatttgctatcagtgagtgctgccttttggcagccatggcttttgaccgctatatggccatatgttccccactccactatgcaacacgaatgagtcgtggggtgtgtgcccatttggcagtggtttcttggacagtgggatgcatggtaggcttgggccaaaccaattatattttctccttagacttctgtggcccctgtgagatagaccacttcttctgtgatctcccacctatcctggcacttgcttgtggggatacatcccataatgaggctgcggtctttgtggtggcaatcctttgcatttctagcccatttttattgatcgttgcttcctatggcagaattctagctgcagtcctggtcatgccctcccctgagggccgccggaaagctctctccacctgttcttcccacttacttgtagtaacgctcttctatggctcaggatctgttacctacttgaggcccaaggctagccactcaccaggaatggataaactgctagccctcttctataccgtggtgacatccatgctcaaccccatcatctacaccctccggaac

SEQ ID NO: 29 - DNA, 708 bp, Mus montanus
cccatgtacttgttcctcggtaatttgtccttcctggagatcctttatacatccacagtggtgccgaaaatgctggagggcttcctgcaggtggcagccatctctgtgactggttgcttgacccagttcttcatctttggttctctagccacagcagaatgcttcctactggctgttatggcatatgatcgcttcttggcaatctgctacccacttcgctatccactcctgatggggcctagatggtgcatggggctggtggtcacagcctggctgtctggcttcatggtagatgaattagttgtggtcctgatggcccagctgaggttctgtggctccaatcgcatcgatcacttttactgtgacttcatgcctttggtggtcctggcttgctcagatcccagagtagcccaggtgacaacatttgttctctctgtagtcttcctcactgttccatttggactgattctgacatcctatgctcgcatcgtggtgactgtgctgagagttcctgctggggccagcaggagaaaggctttttccacatgctcctcccaccttgctgtagtgtccaccttctatggaactctcatggtcttgtacattgtgccctcagctgtccactcccagctcctctccaaggtctttgccttgctctatactgtggtcactcccatcttcaaccccatcatctactccttccggaat

SEQ ID NO: 30 - DNA, 711 bp, Mus montanus
cccaggtacttgtttcttggcaatttgtctttggccgacattgggatcagcacaaccacgatcccccagatggtggtgaacatccagagaaagagaaagaccatcagttacgcaggctgcctcacccaggtctgctttgtcctgatttttgctggatcggagaactttctccttgcagcaatggcttatgaccgttacgcagccatctgccatcccctcaggtacacggccatcatgaacccccacctgtgtgtcctgctggttatgatctccttgtccatcagcacggtggatgccctgctgcacagtctgatgctgctgaggctgtccttctgcacagacctggagatcccccacttcttctgtgaacttgatcaggtgatcacactggcctgttctgacaccctcatcaataacctcctgatatatgtcacagctgggatatttgctggtgttcctctctctggaatcatcttctcttaccttcacattgtgtcctctgtcttgagaatgccatcaccaggaggagtgtataaagccttttccacctgtggctctcacctgtctgtggtctgcttgttctatgggacaatttttggggtgtacattagctctgcagtgactgactcacagagaaaaggtgcagtggcctcagtgatgtactctgtggtccctcagatgctgaaccccatcatctacaccctcagaaac

SEQ ID NO: 31 - DNA, 528 bp, Mus montanus
caagctttggcgtatgacaggtttgtggccatctgtcatcctctgcattatctggtcattatgagccctcgccattgtggcttcttaactttggtgtcatttttgctgagtcttttggactcccagctgcacagtttcatgaccttaaatattaccagcttcaaggatgtggaaatttctaatttcttctgtgacccttctcaactgctgaatctctcctgttccaacaccttctctgataacattgtcaagtattttctgggagccttctatggcctttttcccatctcagggatccttttctcttactacaaaattatttcctccattctgaggatcccctccttaggtgggaagtacaaagccttctccacctgtgggtctcacctggcagttgtttgcttatttttagtgacagcctccacagtgtaccttggatcagttgcatcacattctcccagaaatgatgtggtggcttctctgatgtacactgtggtcacccccatgctcaatcccttcatctgcagt

SEQ ID NO: 32 - DNA, 528 bp, Mus montanus
caagctttggcgtatgataggtttgtggccatctgctaccccctgcactacatggtcatcatgaactcccggcgatgtggattgctgattctggtgtcttggatcatgagtgctcttcattccttgttacaaggtttaatgatgttgagactgtccttctgcacagatttggaaatctcccactttttctgtgaacttaatcacctggtccatcttgcctgctctgacacctttctcaatgaggtggtgatatattttgctgctgtcttgctggctggtggccccctcgctggcatcctttactcttactgcaagatagtctcctccatccatgcaatctcttcagctcagggcaagtacaaagccttctccacctgtgcatctcacctctccgtggtctccttattttattgtacaagcccgggtgtgtacctcagttctgctgtgacccaaaactcacactccactgcaactgcctcggtgatgtacagcgtggtcacccccatgctcaacccctttatctgcagt

SEQ ID NO: 33 - DNA, 528 bp, Mus montanus
caagctttggcgtacgacaggtttgtggccatctgtcacccactgcattatcccagaatcatgagtcagaacctctgtttcctgctagtggttgtgtcctgggtcttatcctctgccaatgcccttttgcacaccctcctcctagcccgtctctctttccttagaggcatcactctgccccacttcttctgtgatctctctgcgttactcaagctatccagctctgacaccaccatcaatcagctggctattctcacggcaggatcagcagttgttaccctgccattcatgtgcattctggtctcatatggccacattggggccaccatcctgagaagaccctccctcaagggcatctgcaaagccttatccacatgtggctcccacctctctgtggtctctgtgtactatggagcagttattgcactctatattgtcccctcatctaatagcactaatgacaaggatattgctgtgtctgtgttgtatactctggtcatccccatgctcaaccccttcatctgcagt

SEQ ID NO: 34 - DNA, 528 bp, Mus montanus
caagctttggcgtatgataggttcttggccatctgttatcccctgcattatacagtcattatgaaccctcgcctctgtggcttctcaattttggtatcatttttgctgagtctcttggactctcagctgcacaatttgatgatcttacaaattaccagtttcaaggatgtggaaatttctagtttcttctgtgacccttctcaacttctgaatctttcctgttctgacaactactctattaatactggcaagtatgttctttttgccctatatagctttttccccatctcagggatccttttctcttactataaaataatttcctccattctgaggatcccatcctcaggggggaagtacaaagccttctccacttgtggctctcacctggcagttttttgcctatttttaggaacaggtactgcagtgtactttggatcagctgtatcacattctcccagggagaatgtggtgtcctcagtaatgtatactgtggtcacccccatgctcaatccctttatctgcagt

SEQ ID NO: 35 - DNA, 528 bp, Mus montanus
caagctttggcgtatgacaggtttgtggccatctgtcaccccctgcattatccaaccattatgaaccctcgattttgtggctttttagttttggtgtcttttttggttagccttttggaatcccagctgcacaatttgattgcattacagtttactactttcaaagatgtaaaaattgctaattttttctgtgacccttctcaggtcctcagtctttcctgttctggcaccttcatcaatatcatagtaatgtattttgttggtgctctatttggtgtttttcccatctcaggaatccttttctcttactataaaatagtttccactattctgagaatcccatcctcaggtgggaaatataaagccttctctacctgtgggtctcacctatcagttgtttgtttattttatggaacaggctttggagtgtaccttggttcagctgtgtcacattcttctagaaaatctgcagtggcctcggtgatgtacacagttgtcacccccatgctcaaccccttcatctgcagt

SEQ ID NO: 36 - DNA, 504 bp, Mus montanus
ggggccattcgctttcccctgcacaatactaccatcatgagccccaagctcggtctcttcctggtggtgctgtcctgggtgctaaccatgttccatgccatgctccataccctgcttatggccagattgtgtttctgtgcagagaacatgattccccattttttctgtgatatgtctgcccttctgaagctgtcctgctccaacactcatgtcaatgagttggtgatattcatcacagcaggcctcattcttctcattccattggtcctcattcttctttcctatgggcacatcgtgtcctccattctcaaggtcccttctgctcgaggtatccataagaccttctccacctgtggctcccatttgtctgtggtgtcactgttctatgggacagtcatcggactctacttatgtccatcagctaataattctactgtgaaagatactgtcatggctctgatgtacacggtggtcactcccatgctcaatccctttatctgcagt

SEQ ID NO: 37 - DNA, 528 bp, Mus montanus
caagctttggcgtatgacagattcctggccatatgtcacccactgcactacactgccatcatgaatcccaggctctgtggtttgctggttctggtgtgctggatcctgagtgtcctgcatgccttgttgcaaagcttaatggtgttgcgactgtccttctgcagagacatagaaatcccccattttttctgtgaactcaaccaggtggtccaacttgcctgttttgacaaccttcttaatgacatagtgatgaattttgcacttgtgctcttggctacttgtcccctcgctggcattctttactcctactccaagatagtctcctccatccgtgcaatctcttcagctcagggcaagtacaaagccttttccacctgtgcctcccacctctctgtggtctccttattttactgcacaggcctgggtgtgtacctcagttctgctgtatcccacagctcacgctccagtgcaacagcctcagtgatgtacaccgtggtcacccccatgctcaaccccttcatctgcagt

SEQ ID NO: 38 - DNA, 357 bp, Mus montanus
cacctttgcaggttgcatctcacagtcctcaagctcgcctgctctgacaccctcatcaacaacatagtggtgttctctatgatcatcgtcctgggtgtcttccctctcagtggcatcctcttctcctactctcagattttctcctccatcctgaggatctcatcagacagaggcaagtacaaagtcttctccacctgtgggtctcacctcctggtggtctccttgttctatggcagtagccttggggtctacctcagttctgtagccacactgtcttctaggatgactctgatggcctcagtgatgtacaccatggtcacccccatgctgaaccccatcatctacaccctccggaac

SEQ ID NO: 39 - DNA, 477 bp, Mus montanus
cccatgtacttgttcctcggtaatttgtccttcctggagatcctttatacatccacagtggtgccgaaaatgctggagggcttcctgcaggtggcagccatctctgtgactggttgcttgacccagttcttcatctttggttctctagccacagcagaatgcttcctactggctgttatggcatatgatcgcttcttggcaatctgctacccacttcgctatccactcctgatggggcctagatggtgcatggggctggtggtcacagcctggctgtctggcttcatggtagatgaattagttgtggtcctgatggcccagctgaggttctgtggctccaatcgcatcgatcacttttactgtcacttcatgcctttggtggtcctggcttgctcagatccccgagtagcccaggtgacaacatttgttctctctgtagtccccctcactgttccattcggactgattctgacatcc

SEQ ID NO: 40 - DNA, 339 bp, Mus montanus
gaggatctatgtgcgagactcaagcgatccaggtcggacaccaccatcaatgaggtgggtattctcacggcaggatcagcagttgttaccctgccattcatgtgcattctggtctcatatggccacatgggggccaccatcctgagaagaccctccctcaagggcatgtgcaaagccttatccacatgtggctcccacctctgtgtggtctctgtgtactatggagcagttattgcactctatattgtcccctcatctaatagcactaatgacaaggatattgctgtgtctgtgttgtatactctggtcatccccatgctcaaccccttcatctgcagt

SEQ ID NO: 41 - DNA, 528 bp, Mus montanus
caagctttggggtatgatagatttgtggccatgtgtcatcctgggcagtatctggtcattatgagccctcgccatggtggcttcctaactttggtgtcatttttgctgagtcttttggactcccagctgcacagtttcatgaccttaaatattaccagcttcaaggatgtggaaatttctaatttcttctgtgacccttctcaactgctgaatctctcctgttccaacaccttctctgataacattgtcaagtattttctgggagccttctatggcctttttcccatctcagggatccttttctcttactacaaaattatttcctccattctgaggatcccctccttaggtgggaagtacaaagccttctccacctgtgggtctcacctggcagttgtttgcttatttttagtgacagcctccacagtgtaccttggatcagttgcatcacattctcccagaaatgatgtggtggcttctctgatgtacactgtggtcacccccatgctcaaccccttcatctgcagt

SEQ ID NO: 42 - DNA, 528 bp, Mus montanus
caagctttggcgtatgacagatttctggccatctgtcatcctctgcattatctggtcattatgagccctcgccattgtggcttcttaactttggtgtcatttttgctgagtcttttggactcccagctgcacagtttcatgaccttaaatattaccagcttcaaggatgtggaaatttctaatttcttctgtgacccttctcaactgctgaatctctcctgttccaacaccttctctgataacattgtcaagtattttctgggagccttctatggcctttttcccatctcagggatccttttctcttactacaaaattatttcctccattctgaggatcccctccttaggtgggaagtacaaagccttctccacctgtgggtctcacctggcagttgtctgcttatttttagtgacagcctccacagtgtaccttggatcagttgcatcacattctcccagaaatgatgtggtggcttctctgatgtacactgtggtcacccccatgctcaacccctttatctgcagt

SEQ ID NO: 43 - DNA, 528 bp, Mus montanus
caagctttggcgtatgacaggttcctggccatctgtcatcctcggcattatctggtcattatgagccctcgccattgtggcttcttaactttggtgtcatttttgctgagtcttttggactcccagctgcacagtttcatgaccttaaatattaccagcttcaaggatgtggaaatttctaatttcttctgtgacccttctcaactgctgaatctctcctgttccaacaccttctctgataacattgtcaagtattttctgggagccttctatggcctttttcccatctcagggatccttttctcttactacaaaattatttcctccattctgaggatcccctccttaggtgggaagtacaaagccttctccacctgtgggtctcacctggcagttgtttgcttatttttagtgacagcctccacagtgtaccttggatcagttgcatcacattctcccagaaatgatgtggtggcttctctgatgtacactgtggtcacccccatgctcaatcccttcatctgcagt

SEQ ID NO: 44 - DNA, 336 bp, Mus montanus
cccatgtatttgtttctcggtaacctgtccttcatggacatctgcttcacaacagtcgttgtgcccaagatgctggcgaatttgctgtcagagacaaagggcatctcctatgtaggctgcctggtccagatgtatttcttcatggcctttgggaacactgatagttacctgctggcctccatggccatcgaccggctggtggccatctgcaaccccttgcactatgatgtggccatgcgcccacaccgctgcctcctcatgctgctgggttcttgcaccatctcccacctgcacgccctcttccgggtgctactcatgtctcgcctctctttctgt

SEQ ID NO: 45 - DNA, 357 bp, Mus montanus
cacctttgcaggttgcatctcacagtcctcaagctcgcctgctctgacaccctcatcaacaacatagtggtgttctctatgatcatcgtcctgggtgtcttccctctcagtggcatcctcttctcctactctcagattttctcctccatcctgaggatctcatcagacagaggcaagtacaaagtcttctccacctgtgggtctcacctcctggtggtctccttgttctatggcagtagccttggggtctacctcagttctgtagccacactgtcttctaggatgactctgatggcctcagtgatgtacaccatggtcacccccatgctgaaccccattatctacaccctccggaac

SEQ ID NO: 46 - DNA, 423 bp, Mus montanus
tggagtcttttggagtcccagctgcacagtttgaggaccttaaatatgaccagcttcagggatgtggaaagttctaatttgttgtgtgacccttctcaaatgctgaatctctcctgttccaacaccttctctgataacattgtcaagtattttctgggagccttctatggcctttttcccatctcagggatccttttctcttactacaaaattatttcctccattctgaggatcccctccttaggtgggaagtacaaagccttctccacctgtgggtctcacctggcagttgtttgcttatttttagtgacagcctccacagtgtaccttggatcagttgcatcacattctcccagaaatgatgtggtggcttctctgatgtacactgtggtcacccccatgctcaacccctttatctgcagt

SEQ ID NO: 47 - DNA, 29 bp, Artificial Sequence
  Description of Artificial Sequence Primer
ccyatgtayttnttyctynsyaayntntc

SEQ ID NO: 48 - DNA, 29 bp, Artificial Sequence
  Description of Artificial Sequence Primer
ccyatgtayttgttyctygsyaayytgtc

SEQ ID NO: 49 - DNA, 27 bp, Artificial Sequence
  Description of Artificial Sequence Primer
rttycknarrswrtanatrawnggrtt

SEQ ID NO: 50 - DNA, 28 bp, Artificial Sequence
  Description of Artificial Sequence Primer
gcactgcagatraanggrttnaratngg

SEQ ID NO: 51 - DNA, 31 bp, Artificial Sequence
  Description of Artificial Sequence Primer
cacaagctttngcntaygayagrtwybtngc

SEQ ID NO: 52 - DNA, 29 bp, Artificial Sequence
  Description of Artificial Sequence Primer
gcactgcagatraanggrttnarcatngg

(19) United States
(12) Patent Application Publication
(10) Pub. No.: US 2002/0132289 A1
(43) Pub. Date: Sep. 19, 2002

(54) Olfactory receptors and their utilizations

(76) Inventors: Jean-Luc Clement, Marseille (FR); Marielle Renucci, Marseille (FR); Valery Matarazzo, Roquevaire (FR); Alain Tirard, Saint-Zacharie (FR); Anne Belaich, Marseille (FR)

Correspondence Address: SCHNADER HARRISON SEGAL & LEWIS, LLP, 1600 MARKET STREET, SUITE 3600, PHILADELPHIA, PA, 19103

(21) Appl. No.: 09/746,284

(22) Filed: Dec. 22, 2000

Related U.S. Application Data
(63) Continuation of application No. PCT/FR99/01495, filed on Jun. 22, 1999.

(30) Foreign Application Priority Data
Jun. 25, 1998 (FR) 98/08094

(51) Int. Cl. C12P 21/02; C12N 5/06; C07K 14/705; C07K 16/28; C07H 21/04

(52) U.S. Cl. 435/69.1; 435/325; 435/320.1; 530/350; 536/23.5; 530/388.22

(57) ABSTRACT

The invention relates to the discovery of new odorant receptors in the marmot, by cloning and by coding gene sequences for these receptors as well as using them for ligand screening and the preparation of biosensors.

### RELATED APPLICATION

This is a continuation of International Application No. PCT/FR99/01495, with an international filing date of Jun. 22, 1999, which is based on French Patent Application No. 98/08094, filed Jun. 25, 1998.

### FIELD OF THE INVENTION

The invention relates to the discovery of new odorant receptors in the marmot, by cloning and by coding gene sequences for these receptors as well as using them for ligand screening and the preparation of biosensors.

### BACKGROUND

The recent discovery of odorant receptors of vertebrae overturns the strategies initially envisaged for the design and production of an artificial nose with physico-chemical sensors. In fact, at the beginning of the 1990's, biologists managed, starting from the odorant epithelium of mammals, to isolate and sequence the first proteins constituting the odorant receptors 3 and, in 1993, the first odorant receptor was expressed 7. Nonetheless, it is admitted that man, who has a limited sense of smell on a relative basis, is capable of differentiating between more than 10,000 odorant molecules and that 1% of his genome is composed of encoding genes for the odorant receptors (1).

It can thus be seen that there is an enormous field of investigation open to researchers in the domain of potential biological sensors. Besides this, it already seems that these biological sensors have a sensitivity which is about 100,000 times higher than the best physico-chemical sensors existing (4, 6). More recent works have shown that these detectors are also sensitive to non-biological molecules (5).

All living organisms depend on sensorial information for their survival. Sensorial perceptions are transmitted by the sense organs which receive the physical stimuli (seeing, hearing, touching) and chemical stimuli (taste, smell). In most species, the perception of chemical stimuli is essential for accomplishing several vital tasks such as finding food, identifying partners, identifying offspring and detecting predators or other dangers. In certain species, the sense of smell also allows communication over distances that can reach several kilometers between individuals, thereby enabling reassembly of the group, attack and defense reactions, and reproduction and suckling activities. The odorant molecules can also induce physiological changes.

In most cases, the odors result from a complex combination of several molecules. This complexity raises interesting questions about the characteristics of the receptors making it possible for animals to recognize a myriad of odorant molecules (estimated at more than 10,000) at concentrations as low as 10⁻¹² M. It seems that recognition is based on a large multigene family of odor receptors comprising several hundreds or thousands of sub-types. These receptors are supposed to contain 7 transmembranous domains, starting from the hypothesis according to which the odorant signals are transducted by cascades of reactions coupled with G proteins in the sensitive olfactory neurones. The transduction results in an increase of second messengers such as cyclic nucleotides or triphosphate inositol and, in their turn, these messengers activate the ion-dependent canals and the phosphorylation of several proteins among which are the odor receptors themselves.

Buck and Axel (3) first of all characterised the odor receptors of rats with the help of amplification techniques (PCR) and degenerate primers corresponding to the most conserved domains of receptors coupled with G proteins. Since these first works, more than 339 receptors have been sequenced, usually partially, among a great variety of species including man, the dog, the mouse, the chicken, two species of fish, two amphibian species and a nematode. However, many species still remain to be studied and it is estimated that more than 1,000 genes (that is 1% of the genome) encode for the super-family of olfactory receptors. The mechanisms subjacent to the olfactory perception are singular and unique in comparison with other sensorial systems and a more extensive study in this domain, which has important implications for identifying these proteins, is necessary.

### SUMMARY OF THE INVENTION

The invention relates to an olfactory receptor including an amino acid sequence selected from SEQ ID No:1 to SEQ ID No:23, or a derivative functionally equivalent thereto. The invention also relates to polyclonal or monoclonal antibodies, nucleic acids, vectors, hosts, membranes, compounds and processes associated therewith.

### BRIEF DESCRIPTION OF THE DRAWINGS

Other advantages and characteristics of the invention will become apparent by reading the following examples concerning the identification and cloning of the olfactory receptors of the marmot, and which refer to the attached drawings in which:

FIG. 1 represents the analysis of PCR products from two types of cDNA ® and T) and 3 primer sets (c-t, 4-1 and 3-2). The reaction products were analyzed by electrophoresis on a 2% agarose gel, as described below in Material and Method. The size of the fragments was estimated by comparison with a standard of known size (right side). The deposits in the tracks marked with an asterisk contain the fragments of the size expected.

FIG. 2 shows the alignment of 14 of the 23 sequences of putative olfactory receptors of the marmot. 14 different sequences (AMOR 1 to AMOR 14) were analyzed using the Clustalw software. The shaded regions indicate the consensus domains containing amino acids almost (.) or totally (*) conserved. The transmembranous domains (DII to DVII), the extracellular loops (E1 to E3) and the intracellular loops (i2 to i3) were defined after determining the hydrophobic domains.

FIG. 3 represents the hydropathy plots of long sequences obtained with the set of c-t primers (AMOR 1 to AMOR 7) and the short sequences obtained with the set of 3-2 primers (AMOR 8 to AMOR 14) were obtained as described in Material and Methods. The long sequences contain 6 regions of high hydrophobicity (peaks) separated by 5 more hydrophilic depressions. The short sequences show only 4 regions of high hydrophobicity and 3 hydrophilic regions. These graphs are compatible with the presence of 6 or 4 transmembranous domains, for the long and short sequences respectively. This architecture is confirmed by the predictions of transmembranous helices by the PHD program.

FIG. 4 represents the analysis of the variability of 14 new non-interrupted sequences of the olfactory receptor of the marmot. The upper graph: variability in the residues calculated for the alignment of FIG. 2. The location of the peaks (the most variable positions) and the overall shape of the curve are independent of the formula used (Wu & Kabat, complexity or number of residues taken into account). The lower graph: average hydropathy index of the aligned sequences. The peaks correspond to the hydrophilic regions (loops) and the depressions to the hydrophobic regions (transmembranous domains). The graph minimizes the hydrophobicity of the fragment 1 to 59 since half the sequences are absent in these positions. While position 210 illustrates the usual variability of the hydrophilic loops shown, position 148 shows the most surprising high variability in a highly hydrophobic region (helicoidal) of the molecule.

FIG. 5 is a dendrogram showing similarities between the olfactory receptors of different species. The sequences of olfactory receptors of other species come from the NCBI data bank. There are five families (noted on the left). The asterisks indicate the sequences for which the percentage of similarity between species exceeds 70%. Abbreviations: H: man; F: fish, C: chicken; N: nematode; B: bee, A: amphibians; D: dog; M: mouse and MM: marmot.

### DETAILED DESCRIPTION

Several works have emphasized the importance of olfaction for the marmot of the Alps (2). Ethological and analytic studies have shown that a group of 40 compounds, produced by the jugal glands, are used to mark territory and identify social groups. Work carried out within the framework of the invention on the olfactory epithelium of the marmot of the Alps was aimed mainly at obtaining a sufficient number of sequences of olfactory receptors to be able to make a significant comparison with the sequences of vertebrae already determined.

A strategy based on the RT-PCR was used for identifying the putative sequences of olfactory receptors of the marmot. Degenerate oligonucleotides corresponding to the sequence of conserved domains in the second transmembranous domain, the second intracellular loop and the 7th transmembranous domain of olfactory receptors were used in pairs as primers for the PCRs starting from the complementary DNA obtained by using the messenger RNA of the nasal epithelium of the marmot.

The research work carried out within the framework of the invention thus made it possible for the first time to identify, clone and sequence new olfactory receptors of the marmot. These receptors are useful for the design and development of biosensors or for the preparation of transfected cells. Thus, these receptors can be associated with artificial membranes which will be used in different biosensors arranged in parallel, each possessing a particular type of receptor, the ensemble being managed by a network software of formal neurones to constitute a detection system of the electronic nose type whose sensors are bio-electronic sensors.

The invention thus concerns a marmot purified olfactory receptor.

The distinction between the tens of thousands of odours depends on a myriad of receptors situated at the surface of the neurone dendrites of the nasal epithelium. By using the nasal epithelium of the marmot of the Alps and different sets of degenerate primers corresponding to consensus sequences of odour receptors, the inventors succeeded in amplifying by reverse-PCR (RT-PCR), cloning and obtaining the partial sequence of 23 new products of encoding genes for odour receptors. After consultation by the Blast software of the NCBI data bank, their translation into sequences of amino acids shows a strong similarity with protein sequences of odour receptors previously reported, and classes them without ambiguity in the same super-family of receptors with 7 transmembranous domains. The transmembranous helicoidal regions III, IV and V, as well as the intra- and extracellular loops have been defined by establishing a hydropathy plot and computer prediction of the secondary structure.

In a first mapping attempt of odour fixation sites, the inventors carried out a variability analysis of the type described by Wu and Kanat (8) on the regions determining the complementarity (CDR) of immunoglobulins. Four principal peaks of variability were located inside the predicted 1st and 3rd extracellular loops, and inside the predicted 4th and 5th transmembranous domains. These positions should thus be part of the specific liaison site for odorant molecules. Comparisons with the sequence of olfactory receptors of other species suggest that the marmot sequences determined in this study belong to three different families.

The invention thus concerns more particularly a purified olfactory receptor constituted by or comprising the sequence of amino acids chosen among those represented in the list of sequences in the appendix under the numbers SEQ ID No:1 to SEQ ID No:23, or a functional derivative equivalent to these. By equivalent derivative of these sequences, we mean the sequences comprising a modification and/or a suppression and/or an addition of one or several amino acid residues, but conserving about 75% and preferably at least about 95% of homology with the sequence from which it is derived. The receptors of the invention present some very conserved regions and some very heterogeneous regions. It is considered that the very conserved regions are those conferring the protein with its receptor property, while the very heterogeneous regions are those conferring each receptor with its specificity. Thus, according to the application envisaged, it is possible to prepare derivatives of the receptors of the invention whose specificity is modified but which remain within the framework of the invention.

Another aim of the invention is polyclonal or monoclonal antibodies directed against at least one receptor of the invention, a derivative or a fragment of these. These antibodies can be prepared by the methods described in the known literature. According to prior art techniques, polyclonal antibodies are formed by the injection of proteins, extracted from the epithelium or produced by genetic transformation of a host, into animals, and then recuperation of antiserums and antibodies from the antiserums for example by affinity chromatography. The monoclonal antibodies can be produced by fusing myeloma cells with spleen cells from animals previously immunized using the receptors of the invention. These antibodies are useful in the search for new olfactory receptors or the homologues of these receptors in other mammals or again for studying the relationship between the receptors of different individuals or species.

The invention also relates to a molecule of nucleic acid comprising or constituted of an encoding nucleic sequence for a receptor such as defined above. In particular, the invention relates to a molecule of nucleic acid comprising or constituted of a sequence chosen among those represented in the list of sequences under the numbers SEQ ID No:24 to SEQ ID No:46, which encode respectively for the receptors whose amino acid sequences are represented in the list of sequences under the numbers SEQ ID No:1 to SEQ ID No:23.

The invention also concerns the nucleotide sequences derived from the above sequences, for example, from the degeneracy of the genetic code, and which encodes for the proteins presenting characteristics and properties of olfactory receptors.

The invention also concerns a vector comprising at least one molecule of nucleic acid above, advantageously associated with adapted control sequences, together with a production or expression process in a cellular host of a receptor of the invention or a fragment thereof. The preparation of these vectors as well as the production or expression in a protein host of the invention can be carried out by molecular biology and genetic engineering techniques well known to the professional.

As an example, a production process of a receptor according to the invention consists of:

transfer of a molecule of nucleic acid of the invention or a vector containing said molecule to a cellular host,

cultivation of said cellular host in conditions allowing production of the protein constituting the receptor,

isolation of said proteins by appropriate means.

As example, a process for expressing a receptor according to the invention consists of:

transfer of a molecule of nucleic acid of the invention or a vector containing said molecule to a cellular host,

cultivation of said cellular host in conditions allowing expressivity of said receptors at the surface of the host.

The cellular host used in the above processes can be chosen among prokaryotes and eukaryotes and particularly among bacteria, yeasts, cells of mammals, plants or insects. Expressivity in eukaryote cells is preferable so that the receptors can undergo the post-translation modifications necessary for their functioning.

A molecule of encoding nucleic acid for an olfactory receptor or a vector according to the invention can also be used to transform animals and establish a line of transgenic animals.

The vector used is chosen as a function of the host into which it is to be transferred. It can be any vector such as a plasmid. Thus, the invention also relates to cellular hosts expressing olfactory receptors obtained in conformity with the preceding processes.

The invention also relates to nucleic and oligonucleotide probes prepared from the molecules of nucleic acid according to the invention. These probes, marked advantageously, are useful for hybridisation detection of similar receptor sequences in other individuals or species. According to prior art techniques, these probes are put into contact with a biological sample. Different hybridisation techniques can be used, such as Dot-blot hybridisation or replica hybridisation (Southern technique) or other techniques (DNA chips). Such probes constitute the tools making it possible to detect similar sequences quickly in the encoding genes for olfactory receptors which allow study of the presence, origin and preservation of these proteins.

The oligonucleotides are useful for PCR experiments, for example, to search for genes in other species or with a diagnostic aim.

As indicated above, the olfactory receptors are proteins with 7 transmembranous domains coupled with G proteins. Attachment of a ligand to a receptor brings about a change in the conformation of the receptor and inside the cell. This signal is transducted through the intermediary of second messengers. Consequently, an aim of the invention is a screening process for compounds capable of constituting ligands of the receptors described above consisting of putting in contact one compound and one or several of said receptors and of measuring by any appropriate means the affinity between said compound and said receptor.

The contact between the compound to be tested and the olfactory receptor or receptors of the invention can be carried out by using the hosts described above and expressing said receptors at least at their surface. It can consist of a line of immortalized cells, olfactory or not, transfected by a vector carrying cDNA making it possible to express at its surface and at a high level a functional recombinant olfactory receptor. If the compound tested constitutes a ligand, its contact with the transformed cells, induces intracellular signals which result from the fixation of said compound on the receptor.

The contact of the compounds to be tested with the receptors of the invention can also be carried out by fixing one or several receptors on one or several membranes. The olfactory receptors of the invention can thus also be integrated with a biosensor. In such a system, it is possible to visualize in real time the interactions between the compound being tested and the receptor. One of the partners of the couple receptor/ligand is fixed on an interface which can contain a matrix covered with aliphatic chains. This hydrophobic matrix can easily be covered with a lipidic layer by spontaneous fusion of liposomes injected into contact with it. Olfactory receptors inserted in the liposornes or vesicles can thus be integrated into the bio-sensors. The ligands are thus analyzed with regard to one or several different olfactory receptors.

The above methods make it possible to determine whether a compound activates or inhibits the receptors. In this embodiment, it is advantageous to use a known ligand which allows measurement by competition.

The invention also relates to a compound constituting a ligand of an olfactory receptor, identified and selected by the above process.

The receptors of the invention find applications in very varied domains such as:

the food processing industry, for detection of aromas, quality control, analysis of samples,

perfumery, for the analysis or comparison of perfumes,

the environment, for detecting toxic substances, such as gases or for trapping odors.

I—Material and Methods

1. Preparation of the tissues.

The olfactory epithelium was removed from a dead wild marmot. During dissection, the head was kept frozen in dry ice. The tissues were kept at −80° C. until used.

2. Isolation of the Messenger RNA.

The frozen tissues were reduced to dust by crushing them with a pestle and mortar. The pestle and mortar were cooled in the dry ice and all the equipment was sterile. The mRNA poly(A)+ was isolated using the Micro-Fast Track Kit (Invitrogen), then tested with the DNA DipStick Kit (Invitrogen).

3. Transcription of the Complementary DNA.

The mRNA poly(A)+ was transcribed in cDNA with the aid of reverse transcriptase then amplified by PCR. In order to increase the production of the first complete strand of cDNA, the cDNA Cycle Kit was used. The reverse transcription was made from 150 ngm of mRNA poly(A)+ using oligo dT primers or random primers. After extraction with phenol/H₂O/EDTA (v/v/v: 1/20/80), the cDNA of the aqueous phase was precipitated in the presence of ammonium acetate and glycogen carrier in iced ethanol at −80° C.

4. PCR.

Three sets of specific degenerate oligonucleotides for olfactory receptors were synthesized to amplify these marmot receptors.

From previous results obtained with the rat (3), two sets of primers were synthesized against the preserved regions of the second and seventh transmembranous domains of the olfactory receptors.

| | |
|---|---|
| Primer 4: | 5′-CC(CT) ATG TA(TC) TTI TT(TC) CT(CT) I(GC)(CT) AA(TC) |
| | (TC) TI IC. |
| Primer C: | 5′-CC(CT) ATG TA(TC) TTG TT(TC) CT(CT) G(GC)(CT) AA(TC) |
| | (TC)TG TC-. |
| Primer 1: | 5′-(AG)TT (TC)C(TG) IA(AG) (AG)(CG)(AT) (AG)TA TAT |
| | (GA)A(AT) IGG (AG)TT. |
| Primer T: | 5′-GCA CTG CAG AT(AG) AAI GG(AG) TTI A(AG) ATI GG. |

These combinations of primers were designed to make it possible to amplify products by the order of 720 pb.

From previous results obtained with the rat (3) and the catfish, the 3rd set of degenerate oligonucleotides was synthesized from the conserved regions of the 2nd intracellular loop and the 7th transmembranous domain.

| | |
|---|---|
| Primer 3: | 5′-CAC AAG CTT TIG CIT A(TC)G A(CT)A G(AG)T (TA)(TC)(TCG) TIG C. |
| Primer 2: | 5′-GCA CTG CAG AT(AG) AAI GG(AG) TTI A(AG)C ATI GG. |

These primer combinations were designed to make it possible to amplify products by the order of 520 pb.

Amplification was carried out in 50 micro liters of a solution containing 5 microliters of cDNA, 2 mM dNTP, 100 pmol of each degenerate primer, 1.5 U of Taq polyinerase (Boehringer Mannheim, Germany), 50 mM KC1, 2.5 mM MgCl₂, 10 mM Tris/HCl pH 8.3 and 0.01 of gelatine. In order to avoid evaporation, the surface of the mixture was covered by 35 microliters of mineral oil (Sigma, France). The PCR was carried out with the aid of a thermocycler (Hybaid, Ornnigene, USA) according to the following protocol: one cycle at 94° C. during 90 sec, 40 cycles at 94° C. during 20 sec, 50° C. during 25 sec and 72° C. during 90 sec, and one cycle at 72° C. during 120 sec.

After the PCR, 5 microliters of the reaction product were analyzed on Seaplaque 2% agarose gel, to verify the presence of the fragment (Tebu). If it was present, the 45 microliters remaining were submitted to electrophoresis and the cDNA was extracted from the agarose gel using the QIARX II kit (Qiagen). The cDNA extract was inserted in the pMOSBlue vector which had been used to infect the competent MOSBlue E. coli cells using the T-vector pMOSBlue kit according to the protocol of the supplier (Amersham). The infected bacteria were then cultivated on a selective medium (Xgal/IPTG).

The recombinant clones were tested by direct on colony PCR. Briefly, each white colony was re-suspended in 10 microliters of TE buffer. The PCR was carried out in 10 microliters of a solution containing 1 microliter of colony suspension, 3 pmoles of each universal primer U19 and T7, 10 mM dNTP, 50 mM KCl and 2.5 mM MgCl₂ in a Tris HCl buffer pH 8.3 with 0.25 U of Taq polymerase. The protocol for the PCR was the following: one cycle at 94° C. during 270 sec, 30 cycles at 94° C. during 30 sec, 48° C. during 30 sec and 72° C. during 50 sec, and one cycle at 72° C. during 120 sec. After the PCR, 10 microliters of the reaction product were analyzed on a 2% agarose gel. The positive clones were cultivated in a liquid LB medium containing 0.1 mg/ml ampicillin.

5. Extraction and Purification of cDNA Fragments.

The plasmidic cDNA was extracted and purified using the Wizard miniprep kit (Promega). The samples were sequenced by Genorne Express (Grenoble, France).

6. Analysis of Sequences.

The comparison of olfactory receptor sequences of the marmot of the invention with sequences available in GenBank/GenPept was carried out using the Blast software on the NCBI server. ClustalW was used to build the multiple alignments and to carry out the phylogenetic analysis. The hydrophobic domains were defined by using a simple hydropathy plot, and the prediction of α-helicoidal transmembranous domains by using the PHD server. Finally, the variability of the 14 marmot sequences aligned, together with their average hydropathy, were determined and transformed into graph form using the Rav3 software. The transmembranous domains were predicted with the Top Pred II software.

II—RESULTS

1. Isolation of the Messenger RNA.

A sample of approximately 2 gm, containing essentially olfactory epithelium and the supporting cartilage was taken from the frozen head of a marmot. This sample was used for purification and the mRNA tests according to the description in the section Material and Methods. In total, 1.95 micrograms of mRNA were obtained. In order to increase the possibilities of cloning the olfactory receptors, half the mRNA obtained was transcribed in presence of the d(T) oligo primer and the other half in presence of the random primer (R).

2. Amplification of Olfactory Receptor Sequences.

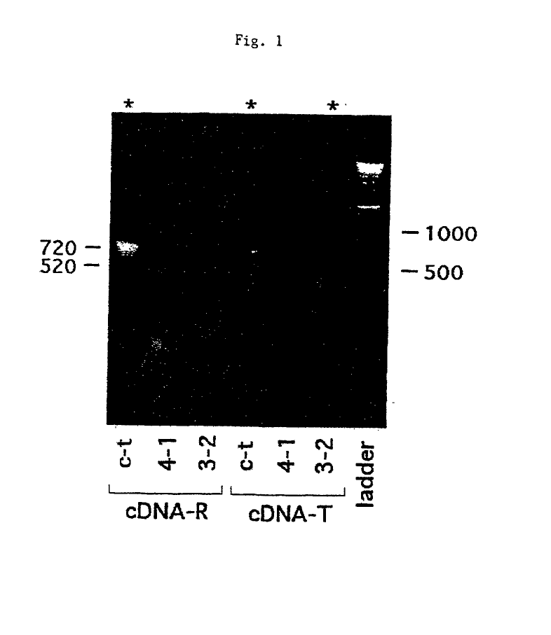

Amplification by PCR was carried out with 150 ngm of mRNA using the three sets of degenerate specific primers (c-t, 4-1, 3-2) described above in Material and Methods. Analysis of the electrophoresis carried out with aliquots of 5 microliters of products from the PCRrevealed single bands of the size expected (FIG. 1). With “T” cDNA, a 520 pb band was obtained with the 3-2 primers and a 720 pb band with the c-t primers. With “R” cDNA, a 720 pb band was obtained using the c-t primers. No band was observed in the three other tracks. In the control PCRs, in which a single primer was used, no band of the length expected was observed. The electrophoresis was repeated using the 45 remaining microliters of the sample, and the fragments of 550 and 720 pb were extracted. Given the diversity of the olfactory receptors, it was considered that the cDNA population in a band was heterogeneous and thus there was no attempt to sequence directly the cDNA fragments amplified by PCR. These fragments were cloned in E. coli as described above.

3. Cloning.

After insertion in the p-Mosblue vector and the infection of competent MOSBlue E. coli, 139 bacterial clones were obtained in total, including 58 from the PCR obtained from the “R” cDNA and the c-t primers (clones R c-t), 31 from the PCR obtained from the “T” cDNA and the c-t primers (clones T c-t) and 50 from the PCR obtained from the “T” cDNA and the 3-2 primers (clones T 3-2). In order to confirm the presence of the expected fragment, we carried out another PCR on each of the 139 clones using the primers corresponding to the vector zones situated on each side of the fragment. Electrophoresis on agarose gel of the PCR products showed that 5 R c-t clones, 10 T c-t clones and 22 T 3-2 clones possessed fragments of the size expected. These 37 positive clones were cultivated again for mass production.

4. Sequencing.

The plasmidic DNA was extracted, purified and sequenced, as described above. The nucleotide sequences were compared with those found in the data banks. Out of the 28 sequences with high scores of similarity with olfactory receptors, 14 were different and uninterrupted (AMOR 1 to 14) and could encode for olfactory receptors. The other 14 sequences were identical (n=8), unusable (n=3) or incomplete for our experimental conditions (116, 153, 159 amino acids). The 14 usable sequences had a single frame open for reading allowing their translation as amino acids. Attribution of the correct reading sequence was confirmed by the similarity of these putative translations with the amino acid sequences of other olfactory receptors available in the Gen Bank/Gen Pept.

The percentage of identical residues in the best alignments spread between 84% (between AMOR4 and a partial sequence of Xenopus laevis access No. #:1617233) and 46% (between AMOR5 and the Rattus norvegicus sequence access No. #:1016362). 7 of the 14 marmot sequences showed the best alignment with different rat receptors, 3 with the same human receptor (access #:AC002988), 3 with the same dog sequence (access #:x89660) and one with the Xenope sequence mentioned above. The average percentage of identical residues was 64%. Seven (AMOR 1-7) of the new marmot sequences were amplified from a couple of primers conceived from the transmembranous domains II and VII and have a length of 234 to 237 residues. Seven other sequences (AMOR 8-14) were obtained with primers conceived from the intracellular loop 2 (i2) and the transmembranous domain VII and contain 176 residues. The percentage of identical residues between these 14 new sequences is comprised between 33% (AMOR 4/AMOR 8) and 79% (AMOR 8/AMOR 11).

5. Structure of the Domain of the Putative Olfactory Receptor of the Marmot.

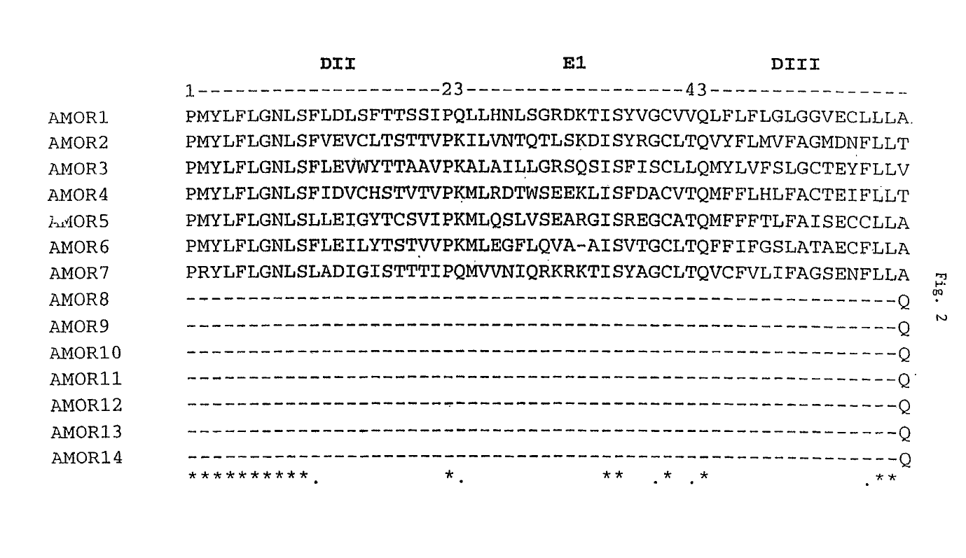

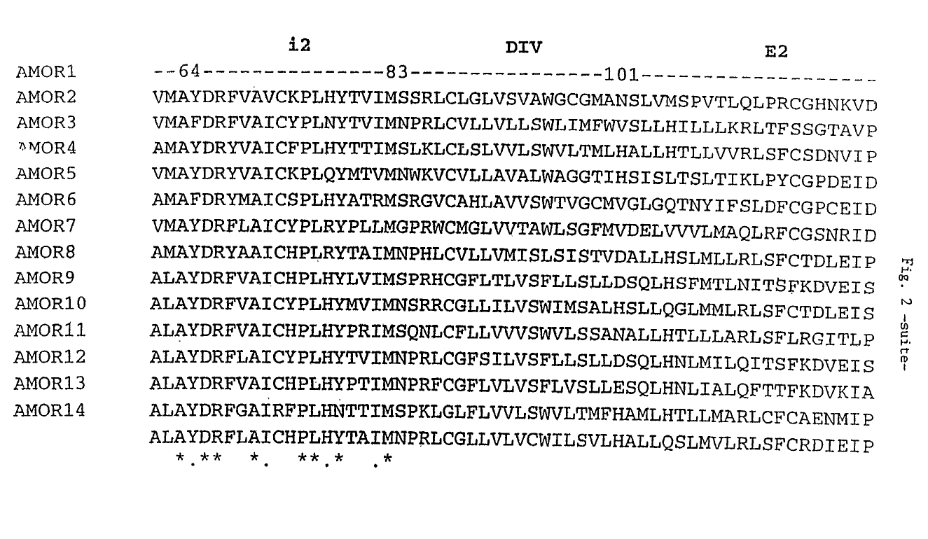

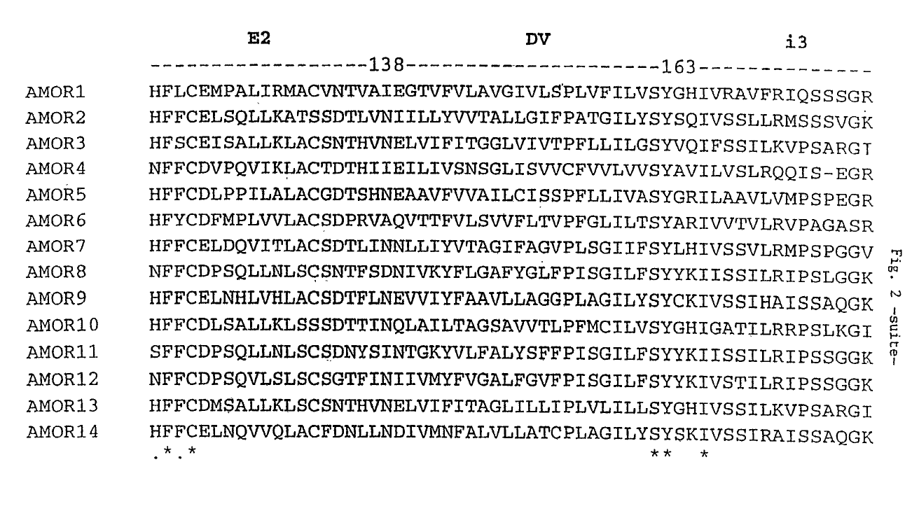

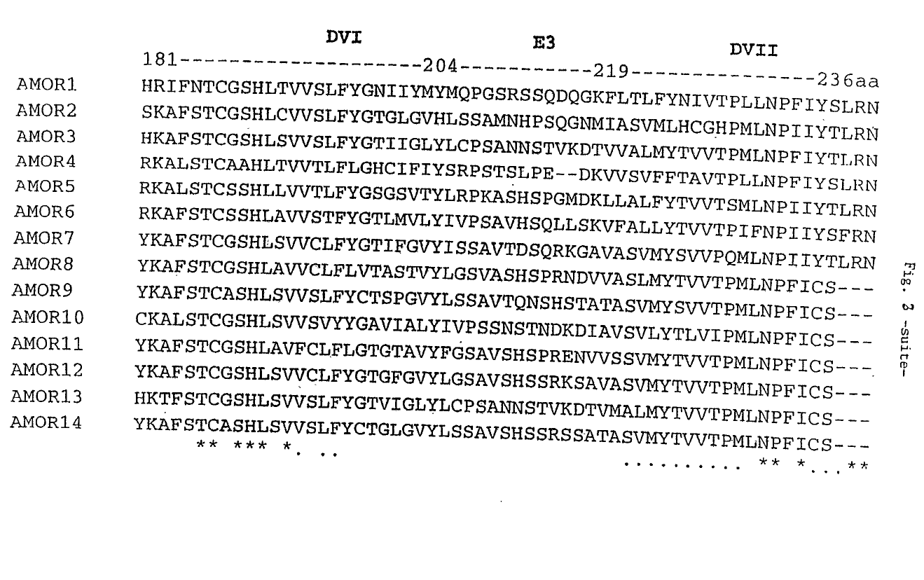

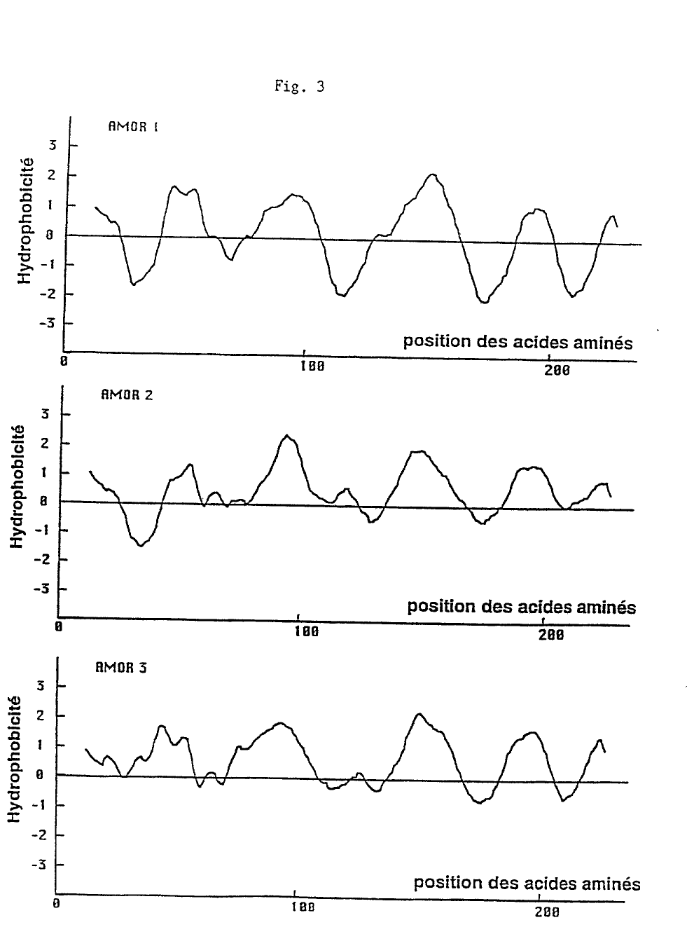

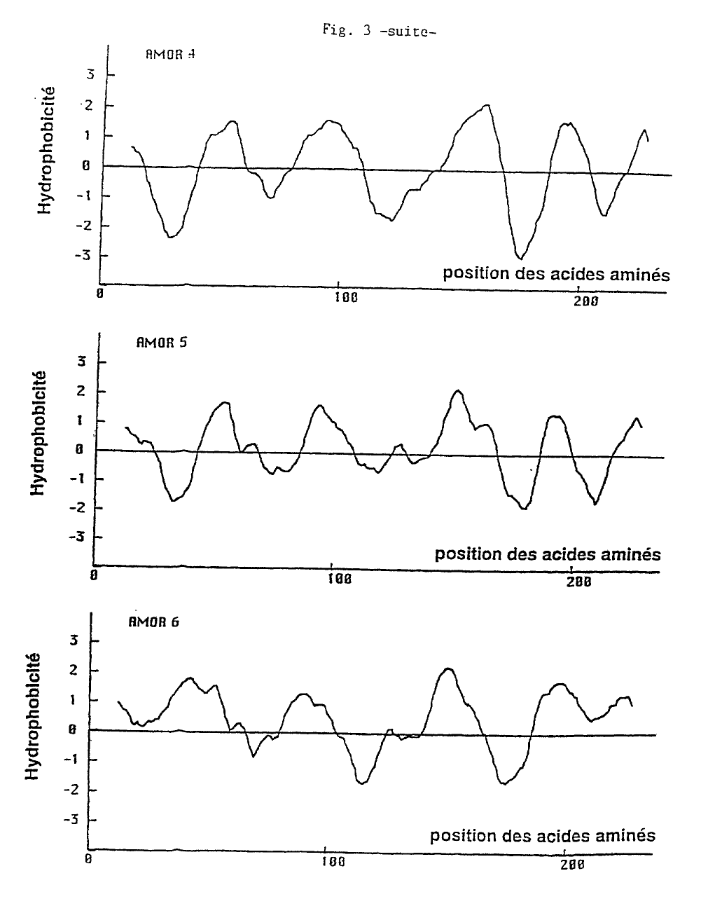

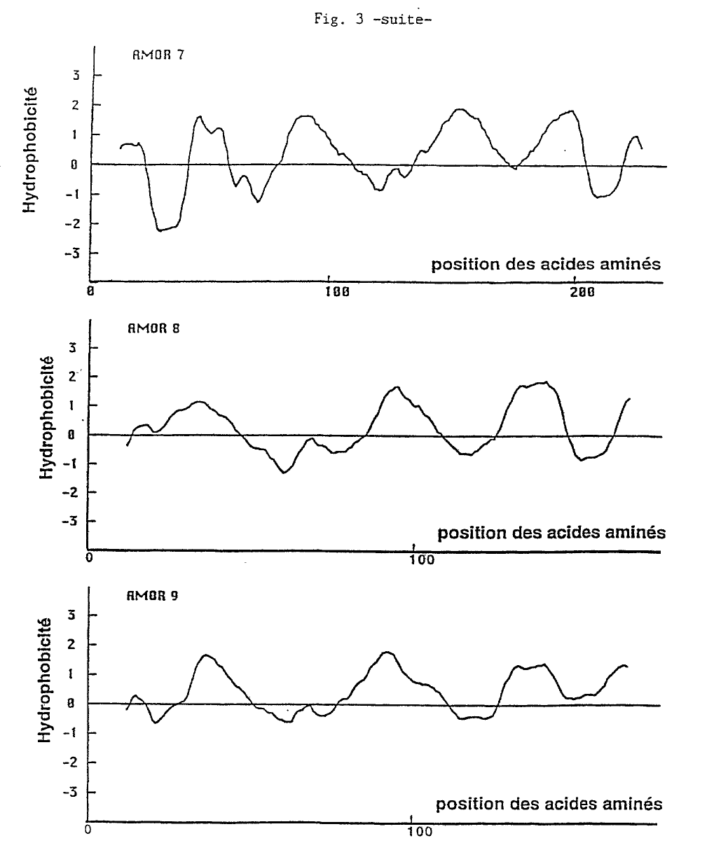

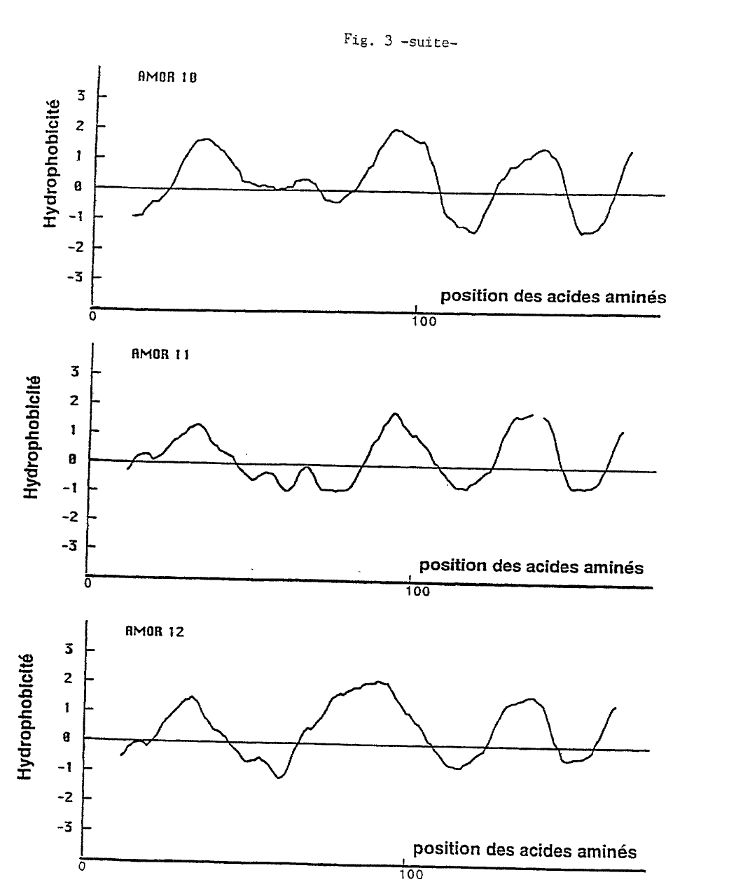

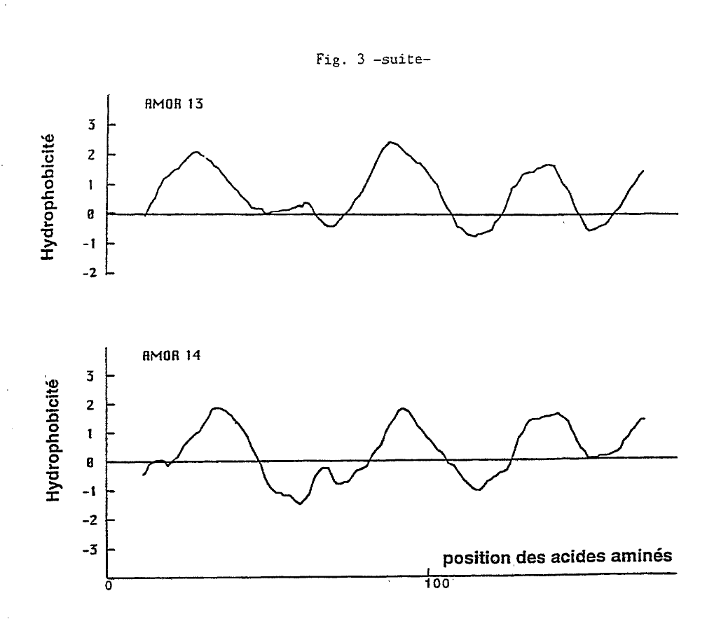

The global homology between the 14 new marmot sequences and the sequences of receptors identified previously leaves little doubt about their belonging to the same super-family of receptors with 7 transmembranous domains. According to the location of the primers used to amplify them, the partial sequences AMOR 1-7 and AMOR 8-14 should present 6 or 4 transmembranous domains respectively. FIG. 3 shows that the hydrophobicity profile of these sequences is compatible with such an organization. In order to define more precisely the a-helicoidal transmembranous regions, the alignment of FIG. 2 was also submitted to the PHD server. 5 transmembranous regions were assigned without ambiguity in the respective regions (38-62), (86-103), (140-164), (186-203) and (216-232), which correspond to the domains DIII, DIV, DV, DVI and DVII in FIG. 2.

The inventors also tried to situate the positions involved in the specific fixation site for odor by applying an analysis introduced previously for molecules which link antigens. Here, the reasoning is that if these olfactory receptors are supposed to link odorant molecules specifically, the residues which constitute the specific linkage site could show more variability than those which are involved in the core structure and in the signaling function.

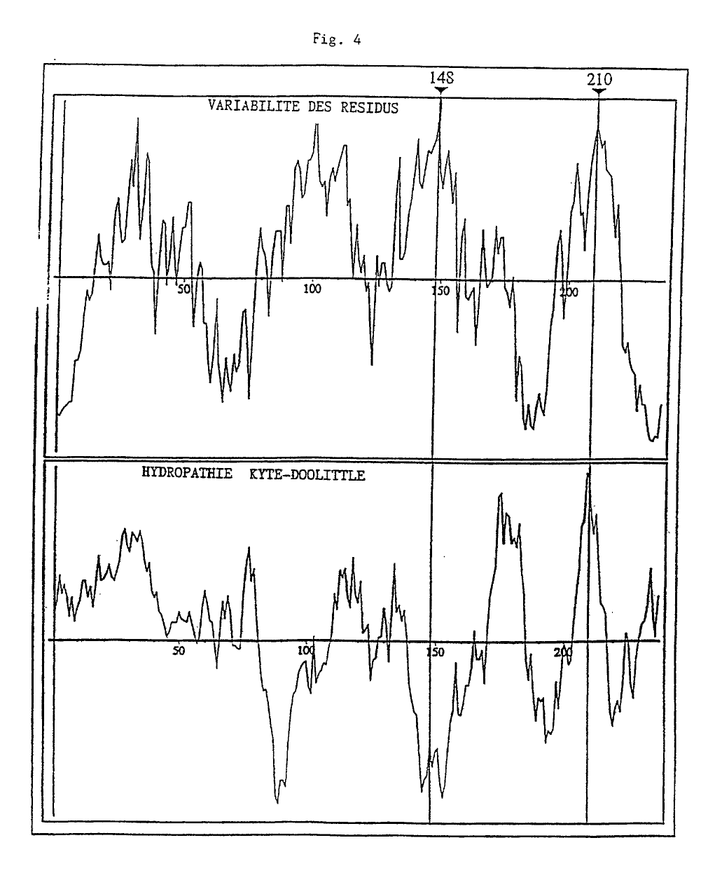

FIG. 4 shows the variability profiles obtained with the alignment of FIG. 2. Four variability peaks are clearly visible. The average hydropathy plot shown in parallel (FIGS. 2 and 4) indicates that they are not only situated inside hydrophilic loops as expected (position 210), but also in hydrophobic regions (e.g. position 148). The center of the most variable segments is situated in positions 30, 100, 148 and 210, the mapping respectively inside the 1st extracytoplasmic loop E1, the 4th and 5th transmembranous regions DIV and DV and the middle of the 3rd extracytoplasmic loop E3. We suggest that the residues in these positions could be implicated in the linking site of unknown odorant molecules corresponding to these receptors. These positions are compatible with the hypothesis according to which the transmembranous regions could assemble in a calyx open to the exterior and able to receive an odorant molecule. Such a model also accords with the fact that many odorant molecules show a hydrophobic character.

6. Structural Classification of Olfactory Receptors.

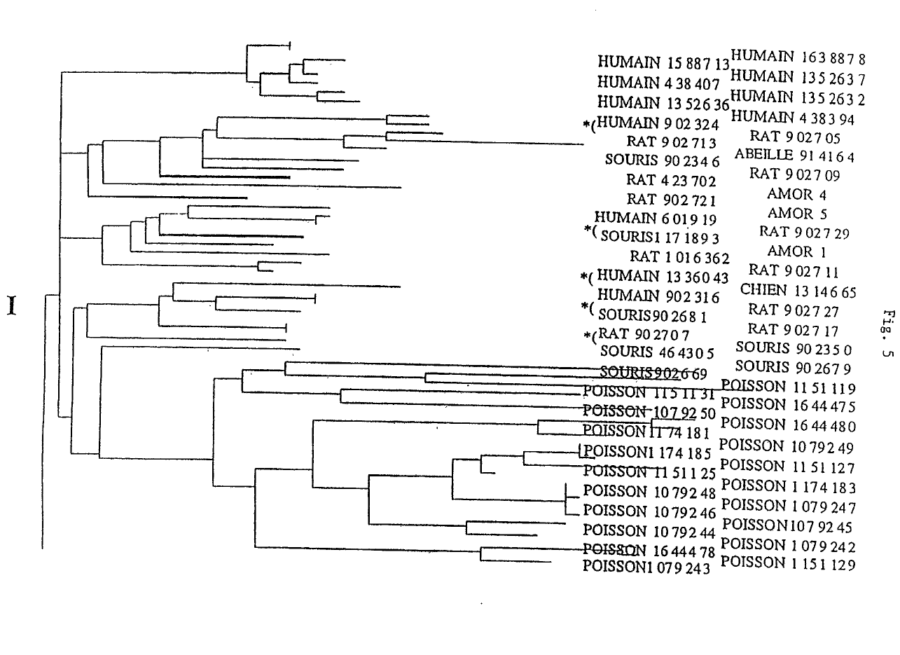

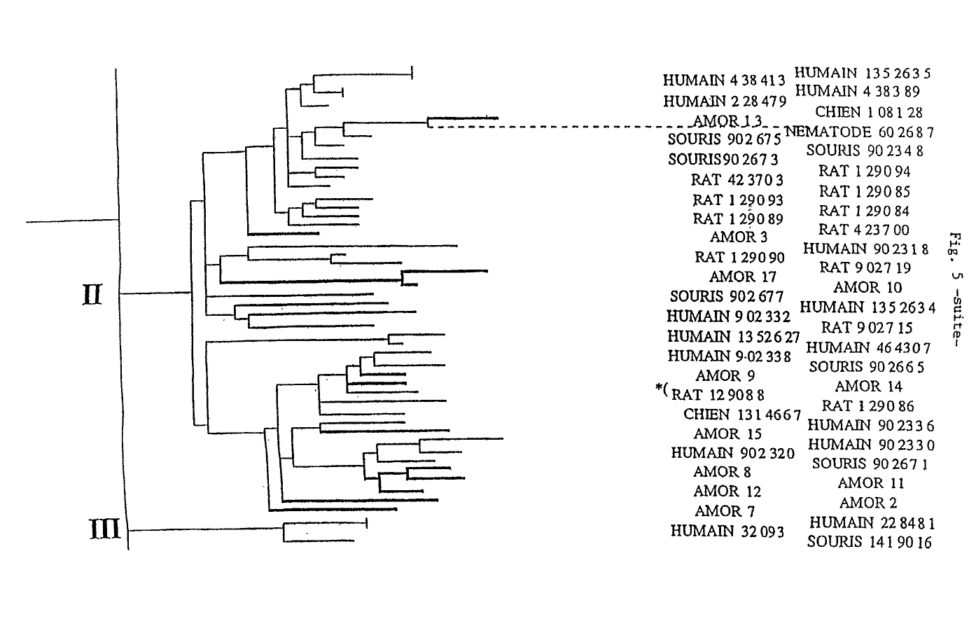

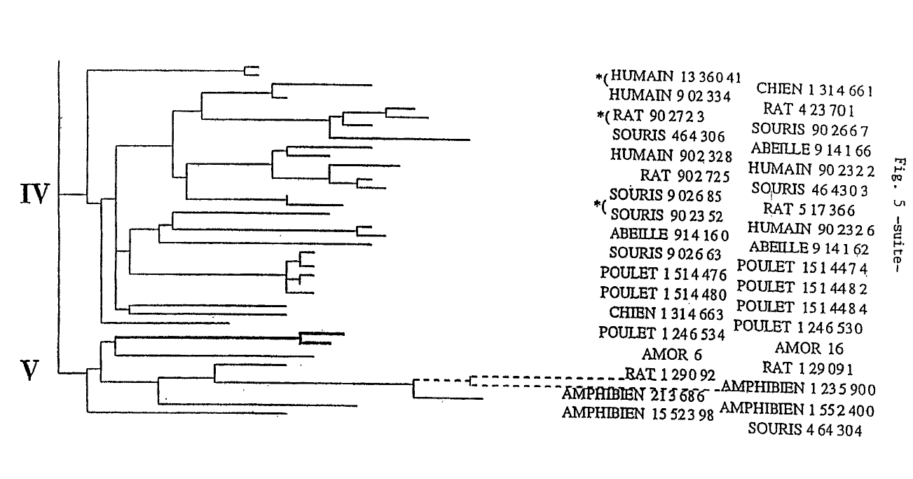

We have tried to classify the cloned receptors of the marmot relative to the sequences described above for other species. FIG. 5 shows a structural classification of 122 olfactory receptors from the EMBLdata bank found in different species as well as the 14 complete sequences and the 3 incomplete sequences identified in the marmot within the framework of the present invention. With the exception of fish receptors, the receptors are not grouped together by species. There are 5 families containing a varied number of receptors. The marmot olfactory receptors were classified in sub-families 1, 2 and 5.12 sequences were classified in the sub-family 2.

The highest percentage of interspecies homologues (over 70% of identical residues) between olfactory receptors was observed in 9 cases indicated by an asterisk: between the rat and the mouse (up to 95%) in 5 cases, between the rat and man (80%) in one case, between the dog and man (up to 85%) in two cases, between the marmot receptor and that of the rat in one case (73%). The homology between human and marmot receptors never exceeded 75% of identical residues.

III—DISCUSSION

The olfactory receptors comprise a large multigene family. Their study requires a combination of approaches. A strategy of reverse PCR with several different primers was used within the framework of the present invention. This approach was crowned with success since 28 putative sequences of olfactory receptors, of which 14 could allow comparative analysis, were obtained. It is possible to obtain more sequences by simply changing the PCR conditions. The family of genes cloned within the framework of the present invention encode olfactory receptors for two reasons. On the one hand, the hydropathy plots of sequences are in agreement with the receptors of the super-family of receptors with seven transmembranous domains. On the other hand, comparison with the sequences in data banks shows a strong degree of similarity with the olfactory receptors previously identified.

The potential sites for ligand recognition on the putative olfactory receptors of the marmot have been identified. Since olfaction requires the specific recognition of a great variety of odorant molecules, it was postulated that the liaison site of the olfactory receptor with its ligand would present a greater variability between residues than the other parts of the sequence responsible for the core structure and the function of transduction. The greatest variability was observed within two transmembranous domains (DIV and DV) and within two extracellular loops (E1 and E3). It was therefore concluded that these regions could be involved in the recognition of the ligand.

The presence of a deep liaison site in the transmembranous calyx is not a property specific to receiving olfactory receptors but is common among receptors with 7 transmembranous domains of biogenic arnines.

The principal interaction site between the receptors with 7 transmembranous domains and the related G protein is the third intracellular loop. For the sequences presented here, the most conserved segment is located between positions 180 and 193, that is to say the end of this loop and the beginning of the 6th transmembranous domain.

The results obtained indicate a remarkable analogy between the olfactory receptor of the marmot and the olfactory receptor of the rat. The length (18 residues) of the 3rd intracellular loop (B) was short. The IVSSI consensus sequence (or a close sequence) was at the N-terminal end of the 3rd intracellular loop in 75% of clones of the invention. The third intracellular loop is rich in Serine residues and can thus constitute phosphorylation sites for GRK. The receptors with 7 transmembranous domains are classified into several groups. The olfactory receptors are supposed to belong to the group 1, which is characterised by the presence of a strictly conserved DRY sequence of the N-terminal side of i2. The DRY sequence is present in 4 of the clones of the invention but is replaced by a DRF sequence in the remaining 10.

The recognition of the same odors by different species brings up an interesting question. It can be expected that these species have autologous receptors. Using the clustalW software (FIG. 5), the inventors tried to determine whether certain olfactory receptors of the marmot were bona fide autologues of olfactory receptors of other species, in particular other rodents. For the receptors coupled with G proteins, the identity percentages between the autologous receptors of different species ranged from 68% (for the CSN receptor, between the dog and man) to 98% (for the cannabinoid receptor of the rat and man). Olfactory receptors with percentages of similitude of this order were observed between the rat and the mouse, the rat and man, and the dog and man. A single marmot olfactory receptor showed a similitude percentage of this order with a rat receptor (AMOR14 73%). In general, we found few close homologues. This discovery could indicate that either the number of olfactory receptors was too small to allow identification of real autologous receptors, or the percentage of similarity between autologous olfactory receptors can become lower than 68%.

Another alternative could be that wild animals express receptors for a greater number of odors than laboratory animals. The marmot of the Alps (Marmota marmota) was chosen as a model in this study based on the hypothesis that, given the importance of olfaction in its survival in the wild, its olfaction must be highly developed. The marmot of the Alps marks out its territory with secretions produced by its jugal glands. In addition, for this animal, the sense of smell is of greatest importance because this species possesses a very high sociability level: it lives in family groups formed by a pair of resident reproductive adults and their offspring of several successive litters which stay in their natal group until the age of 2 years or more. Each marmot has a combination of different odorant molecules which members of the same group or of a different group can sense.

Contrary to other sensor systems, the olfactory system requires a myriad of different receptors. Since mammals are supposed generally to have about a thousand genes, the clones identified in this study probably represent only a part of the family of olfactory receptors of the marmot. In addition to the contribution to the number of receptors identified, our results also support the existence of autologous receptors between species and the notion that the local variability observed in certain transmembranous domains could be capital for the specificity of a receptor. How even a thousand receptors could be able to distinguish among the tens of thousands of odors found in nature is not yet clarified. The final confirmation of the nature and olfactory specificity of these receptors will not be possible until the entire sequence has been obtained and the specific liaison with one or several odorant molecules demonstrated.

### BIBLIOGRAPHIC REFERENCES

(1) Axel R. (1995). The molecular logic of smell. Scientific American October 154-159.

(2) Bel M-C., Porteret C. and Coulon J. (1995). Scent deposition by cheek rubbing in the Alpine marmot (Marmota marmota) in the French Alps. Can. J Zool., 73. 2065-2071.

(3) Buck L. and Axel R. (1991). A novel multigene family may encode odorant receptors: a molecular basis for odor recognition. Cell. 65, 175-187.

(4) Cornell B. A., Braach-Maksvytis, V. L. B., King L. G., Osman P. D. J., Raguse B., Wieczorek L. and Pace R. J. (1997). A biosensor that uses ion-channel switches. Nature 387, 580-583.

(5) Kinoshita. T. (1995) Biomembrane mimetic systems. Prog. Polym. Sci., 20, 527-583.

(6) Mielle P. (1998). Une technique de pointe au service du contrôle de la qualité aromatique. Biofuture, 174, cahier n° 99.

(7) Raming K., Krieger J., Strotmann J., Boekhoff I., Kubick S., Baurnstark C., and Breer H. (1993). Cloning and expression of odorant receptors. Nature, 361, 353-356.

(8) Wu T. T., and Kabat E. A. (1970). An analysis of the sequences of the variable regions of Bence Jones proteins and myeloma light chains and their implications for antibody complementarity. J. Exp. Med., 132, 211-250.

What is claimed is:

1. A purified olfactory receptor of a marmot.

2. An olfactory receptor comprising an amino acid sequence selected from SEQ ID No:1 to SEQ ID No:23, or functionally equivalent derivative thereof.

3. The receptor according to claim 2, comprising an amino acid sequence having about 75% homology with an amino acid sequence selected from SEQ ID No:1 to SEQ ID No:23.

4. The receptor according to claim 2, comprising an amino acid sequence having about 95% homology with an amino acid sequence selected from SEQ ID No:1 to SEQ ID No:23.

5. The receptor according to claim 2, comprising an amino acid sequence selected from SEQ ID No:1 to SEQ ID No:23 in which at least one heterogeneous region is modified.

6. An antibody directed against at least one receptor according to claim 1 or a derivative or a fragment thereof.

7. A nucleic acid fragment comprising a nucleic sequence encoding a receptor according to claim 2.

8. A nucleic acid fragment according to claim 7, comprising a sequence selected from SEQ ID No:24 to SEQ ID No:47.

9. A vector comprising at least one nucleic acid fragment according to claim 7, operably connected to at least one regulatory sequence.

10. A method for making a receptor comprising:
a) transferring a molecule of nucleic acid according to claim 7 into a cellular host,
b) cultivating said cellular host under conditions suitable for production of the receptor, and
c) isolating said receptor.

11. A method for making a receptor comprising:
a) transforming a cellular host with the vector of claim 9;
b) cultivating said cellular host under conditions suitable for production of the receptor, and
c) isolating said receptor.

12. An expression process of a receptor in a host, comprising:
a) transforming a host with the nucleic acid fragment of claim 7; and
b) cultivating said host under conditions suitable for expression of said receptor at a surface of the host.

13. An expression process of a receptor in a host, comprising:
a) transferring a vector according to claim 9 into a host,
b) cultivating said host under conditions suitable for expression of said receptor at a surface of the host.

14. A host transformed by the nucleic acid fragment according to claim 7.

15. A host transformed by a vector according to claim 9.

16. A method for screening compounds which are capable of binding to the receptor according to claim 2, comprising:
a) contacting a compound and at least one receptor; and
b) measuring affinity between said compound and said receptor.

17. A membrane on which at least one receptor comprising an amino acid sequence selected from SEQ ID No:1 to SEQ ID No.:23, or a functionally equivalent derivative thereof is immobilized in said membrane for use in the method of claim 16.

18. A compound constituting a ligand of an olfactory receptor, identified and selected by the process according to claim 13.

19. Utilization of a receptor according to claim 2, for detection of aromas, quality control, sample analysis, analysis or comparison of perfumes, detection of toxic substances, or trapping of odors.

20. Utilization of a host according to claim 14, for detection of aromas, quality control, sample analysis, analysis or comparison of perfumes, detection of toxic substances, or trapping of odors.

21. Utilization of a membrane according to claim 17, for detection of aromas, quality control, sample analysis, analysis or comparison of perfumes, detection of toxic substances, or trapping of odors.
